(19) United States
(12) Patent Application Publication
(10) Pub. No.: US 2026/0165946 A1
(43) Pub. Date: Jun. 18, 2026

(54) USE OF PLANT EXTRACTS IN COSMETICS

(71) Applicant: ROBERTET S.A., Grasse (FR)

(72) Inventor: Anthony PEGARD, Grasse (FR)

(21) Appl. No.: 19/532,125

(22) Filed: Feb. 6, 2026

Related U.S. Application Data
(63) Continuation of application No. 17/270,998.

(30) Foreign Application Priority Data
Sep. 13, 2018 (FR) 1858218

(51) Int. Cl. A61K 8/9789; A61Q 7/00

(52) U.S. Cl. CPC A61K 8/9789; A61Q 7/00

(57) ABSTRACT

The cosmetic use of at least two extracts obtained from two vegetables selected from Piper cubeba, Piper nigrum and a plant of the genus Schinus in particular the species Schinus terebenthifolius, is to promote hair growth.

### CROSS-REFERENCE TO RELATED APPLICATIONS

This application is a Continuation of U.S. patent application Ser. No. 17/270,998 filed 24 Feb. 2021, which is a 35 U.S.C. § 371 National Stage patent application of PCT/FR2019/052128 filed 13 Sep. 2019, which claims the benefit of French patent application 1858218 filed 13 Sep. 2018, the disclosures of which are incorporated herein by reference in their entirety.

### TECHNICAL FIELD

The present disclosure relates to the cosmetic use of vegetable extracts to promote the hair growth.

In humans, one tries to promote the hair growth on different portions of the body for mainly aesthetic reasons.

Within the scope of the present disclosure, the term «hair» will designate either the head hair or the body hair, such as beard hairs.

Body hair and head hair are made by the assembly of cells produced in the follicle by keratinization, the hair follicle being the cavity in which they originate.

The main difference between the body hair and the head hair lies in their growth. While the head hair grows indefinitely, continuously, the body hair stops its growth once the maximum magnitude has been reached, then is replaced by a similar one.

The vegetables are a well-known source of extracts and active ingredients allowing stimulating the hair growth.

Thus, the object of the document KR 100848800 B1 is a cosmetic composition based on Brazil pepper oil (also called pink berries), extracted from a plant of the species Schinus terebinthifolius to, among other properties, treat the head hair loss and promote its growth.

The document JP H10265340 A describes derivatives of 1,3-benzodioxole preferably isolated from plants such as those of the family Piperaceae, and in particular of the species Piper retrofractum vahl and Piper nigrum L., to stimulate the head hair growth and prevent alopecia.

According to the document N. Hirata and al., Biol. Pharm. Bull. 30 (12) (2007) pp 2402-2405, the cosmetic use of Piper nigrum extracts is known to treat both alopecia, by inhibition of testosterone-5-alpha-reductase, and the depigmentation of head hair by stimulation of the melanogenesis.

The hair follicle constitutes the fundamental biological unit, directly involved in the development and the maintenance of the hair system. It brings together different structural entities such as the dermal papilla, the sebaceous gland, the epithelial sheaths, innervation and vascularization, involved separately or jointly in the head hair development cycle.

### SUMMARY

Compared to the prior art above, the disclosure provides vegetable extracts which act directly on the hair follicle and more specifically in the dermal papilla, allowing promoting the hair growth and this, regardless of the state of the treated subject, in particular whether or not he is suffering from alopecia or any other form of hair loss or weakness.

Thus, the present disclosure relates in particular to the cosmetic use of at least two vegetable extracts to promote the hair growth. These extracts are obtained from at least two vegetables selected from Piper cubeba, Piper nigrum and a plant of the genus Schinus.

Indeed, surprisingly, it has been found that a combination of at least two vegetable extracts, advantageously of two or three vegetable extracts, selected from the extracts obtained from at least two plants selected from the species Piper cubeba, the species Piper nigrum and the plants of the genus Schinus have an action promoting the hair growth. Among the species of the genus Schinus, the species Schinus terebenthifolius is preferred, without, however, the disclosure being restricted thereto. As will be demonstrated later, a synergistic effect of the extracts therebetween on the hair growth is observed.

Before exposing the disclosure in more detail, some terms used in the present text are defined.

Within the scope of the present disclosure, the term «promoting growth» means an increase in the density and/or the thickness of the hair.

The species Piper cubeba is also called cubeb; the species Piper nigrum is also called black pepper or black pepper tree; and the species Schinus terebenthifolius is still known under the names of pink pepper and pink berries.

The terms “composition”, “combination” or “association” will be used interchangeably to designate a mixture of at least two extracts used according to the disclosure. The term “formulation” will designate any mixture of at least two extracts, as defined in the present disclosure, with any other material(s), for example one or more other active ingredient(s) which are different from the extracts of the disclosure contributing to the desired effect or providing another activity; one or more additive(s) such as a perfume, a preservative, an antioxidant; one or more solvent(s); one or more excipient(s); one or more adjuvant(s); and this, regardless of the form of the said material(s), in particular solid, liquid, and regardless of the natural or synthetic origin thereof. Within the scope of the disclosure, this or these material(s) is/are dermatologically or cosmetically acceptable.

In the description the terms “the extract is obtained from a plant”, “the extract is obtained from a vegetable”, and “extract of” have the same meaning.

The effectiveness of the vegetable extracts of the disclosure on the density and/or the thickness of the hair is measured compared to a control by methods which are well known to those skilled in the art such as, for example, using a videomicroscope.

It is also possible to evaluate the effectiveness of the combinations of extracts by studying the hair density (total number of hairs per cm²), the hair growth speed (in mm per day) or the variation in the thickness of the hairs by scoring or else by studying the effect of the compositions on cells of the dermal papilla of human follicles (HFDPC, Human Follicle Dermal Papilla Cells) and more particularly by the effect of the compositions on the synthesized quantity of vascular endothelial growth factor (VEGF).

Within the scope of the present disclosure, the vegetable extracts of the present disclosure are extracts which are used in perfumery, such as essential oils, concretes, resinoids, absolutes, supercritical CO₂ extracts or even extracts obtained with polar solvents such as alcoholic, hydro-alcoholic, glycolic or glycerin extracts. They are prepared according to techniques which are well known to those skilled in the art.

In one particular aspect, the disclosure relates to the cosmetic use of an extract of Piper nigrum and an extract of a plant of the genus Schinus, of an extract of Piper cubeba and an extract of a plant of the genus Schinus and an extract of Piper nigrum and an extract of Piper cubeba. In another aspect, the disclosure concerns the cosmetic use of an extract of Piper nigrum, an extract of Piper cubeba and an extract of a plant of the genus Schinus to promote the hair growth. In these combinations of two or three extracts, the plant of the genus Schinus is advantageously the species Schinus terebenthifolius.

### DETAILED DESCRIPTION

Within the scope of the present disclosure, the extract(s) are present in an effective quantity to promote the hair growth.

Those skilled in the art will know how to define the stoichiometry of each of the vegetable extracts present in the composition of the disclosure.

In a particular implementation of the disclosure, the extracts may be present in identical weight (1/1 for two extracts or 1/1/1 for three extracts); in another implementation the extracts may be present in different quantities.

In a particular aspect, the vegetable extracts can be combined with at least one other compound.

Thus, the disclosure also concerns a cosmetic formulation comprising at least two vegetable extracts as defined above.

A cosmetic formulation of the disclosure can comprise at least one odorous compound, at least one antioxidant and/or at least one solvent.

Odorous compounds are well known to those skilled in the art in the field of cosmetology. These compounds are present in a variety of chemical classes, but are generally compounds which are volatile and insoluble in water. These odorous compounds are found in vegetable extracts such as essential oils, absolutes, resinoids, CO₂ extracts or are synthesis products. These compounds are present in sufficient quantity to provide a pleasant odor which can be perceived by a consumer.

The solvents which are likely to be used are also well known to those skilled in the art, mention may be made, by way of example, of isopropyl myristate, benzoyl benzoate, triethyl citrate or diethyl phthalate, polar solvents, water, ethanol, glycerol, glycerinated water, glycol ethers such as propylene glycol, butylene glycol, dipropylene glycol (DPG), used alone or in mixtures.

The antioxidant(s) which are likely to be used are also well known to those skilled in the art, tocopherol, butylated hydroxytoluene (BHT), the Trolox may be mentioned by way of examples.

In a particular implementation of the disclosure, the combination of extracts preferably represents from 10 ppm to 20% by weight relative to the total weight of the cosmetic formulation. According to another particular implementation, the combination of extracts represents from 1% to 35% by weight relative to the total weight of the cosmetic formulation.

The present disclosure also concerns the use of the combination of extracts in a cosmetic formulation comprising said combination according to the disclosure. As previously indicated, such a cosmetic formulation comprises any other dermatologically or cosmetically acceptable material.

This or these dermatologically or cosmetically acceptable material(s) are well known to those skilled in the art.

The cosmetic formulation according to the present disclosure can be, for example, a cosmetic or dermatological product, in the form of an ointment, a lotion, a foam, a cream, a gel, a solution, an oil-in-water or water-in-oil emulsion, an unguent, a body oil, etc.

The cosmetic formulation according to the present disclosure can also take the form of a lotion or a solution in which the combination according to the disclosure is in an encapsulated form. The combination according to the disclosure can be incorporated in vectors of the liposome, glycosphere, cyclodextrin type, in chylomicrons, macro-, micro-, nano-particles as well as the macro-, micro- and nano-capsules and also be absorbed on powdery organic polymers, talcs, bentonites and other mineral supports.

The cosmetic formulation according to the present disclosure can take the form of a gel comprising appropriate excipients such as cellulose esters or other gelling agents, such as carbopol, sepinov (polyacrylate), guar gum, etc.

The cosmetic formulation according to the present disclosure can also contain additives or adjuvants which are common in cosmetology such as, for example, antimicrobial agents but also extraction or synthesis lipids, gelling and viscosifying polymers, surfactants and emulsifiers, water- or liposoluble active ingredients, plant extracts, tissue extracts, marine extracts, synthesis active ingredients.

The cosmetic formulation according to the present disclosure can also comprise other complementary active ingredients selected for their action, for example for the moisturizing effect, the anti-aging effect, the antimicrobial activity, the antioxidant activity, the wound healing effect, the tightening effect, the anti-wrinkle effect, the chelating activity, the complexing and sequestering activity, the soothing effect, the anti-redness effect, the emollient activity, the activity participating in the cell renewal, but also the sun protection, the anti-irritant activity, the cellular nutrition, the cellular respiration, anti-seborrheic treatments, the skin tone.

Those skilled in the art will know how to define the effective quantities to be used depending on the type of cosmetic product comprising the synergistic composition according to the disclosure; for example, the vegetable extracts conventionally used in the rinse-off products (for example shampoos, hair lotions) will be present in a quantity of approximately 1% by weight relative to the total weight of the cosmetic formulation.

When the cosmetic formulation according to the present disclosure contains complementary active ingredients, these are generally present in a sufficiently high concentration so that they can exercise their activity.

The cosmetic formulations according to the present disclosure are preferably used daily and applied one or several time(s) a day.

This use can be repeated until obtaining an acceptable result by the user.

The formulations according to the present disclosure are very well tolerated, they exhibit no toxicity and the application thereof on the skin, for prolonged periods of time, does not involve any systemic effect.

The present disclosure also relates to a method for promoting the hair growth comprising the selection of a skin area on which the hair growth and/or regrowth is desired, and the application on said area of a cosmetic formulation, according to the disclosure, in a sufficient quantity to promote the hair growth.

The selected skin area can be located on the scalp, the face, the legs, the pubis, the torso, the arms or the armpits.

The present disclosure is illustrated in a non-limiting manner by the following examples.

### Example 1: Cell Viability Test

A cell viability test using tetrazolium salt MTT (3-(4,5-dimethylthiazol-2-yl)-2,5-diphenyltetrazolium bromide) is carried out. The MTT test is a method for counting living cells, which is well known to those skilled in the art.

For this, the cells are incubated for 3 hours with the medium containing 1 mg/ml of MTT, before dissolving the formazan crystals formed with DMSO and reading the absorbance at 570 nm. The cell survival rate is expressed as a percentage relative to the negative controls. It is measured in the presence of an extract quantity of according to the disclosure corresponding to their EC50 (see Example 2).

The results are shown in Table 1 below.

TABLE 1
MTT test of vegetable extracts used in combinations
according to the disclosure

| | % cell survival |
|---|---|
| Ingredient (proportion) | at EC50 |
| Black Pepper Essential Oil | 100 |
| Cubeb pepper CO₂ extract | 89 |
| Pink berry CO₂ extract | 97 |
| Black Pepper/Cubeb mixture (1/1) | 93 |
| Black pepper/Pink berry mixture (1/1) | 100 |
| Cubeb/Pink berry mixture (1/1) | 90 |
| Cubeb/Pink berry/Black pepper mixture | 92 |
| (1/1/1) | |

It appears that the extracts according to the disclosure do not exhibit toxicity.

### Example 2: Study of the Quantity of VEGF Produced by the Cells in the Presence of Different Vegetable Extracts

Several factors are responsible for the head hair growth, one of the most important being the VEGF rate present in the dermal papillae of the scalp.

This cytokine is best known for the angiogenic activity thereof. It increases the vascular permeability and thereby the blood circulation of the tissue, which results in the hair growth (Yano and al., «Control of hair growth and follicle size by VEGF-mediated angiogenesis», J. Clin. Invest., 2001, 107, p. 409-417).

VEGF allows, by increasing the blood circulation of the hair tissue, a better nutrition of the base of the follicle. The observations linking the VEGF to the hair growth are numerous.

The test is based on the study of the effect of the product tested on the human follicle dermal papilla cells (HFDPC) and the study allows evaluating the quantity of vascular endothelial growth factor (VEGF) in the presence of different compositions according to the disclosure.

In vivo model tests on HFDPC cells were necessary. They are primary lines of human follicular dermal papilla cells found at the base of the hair follicles. They are cultured in complete medium composed of dermal papilla follicle cell growth medium (FDPCGM), fetal calf serum 4%, bovine pituitary extract 0.4%, basic growth factor of recombinant human fibroblasts 1 ng/ml and recombinant human insulin 5 μg/mL.

For the experiments, the HFDPC are seeded in 24-well plates, at a density of 16,000 cells/cm² and incubated for 24 hours at 37° C., 5% CO₂. The cells are then placed in the presence of a medium containing the different raw material concentrations (between 0 and 100 ppm) for an incubation of 24 hours. After this time, the cell supernatants are collected in microtubes for protein assays; while cytotoxicity tests are carried out on the cells (see Example 1).

The VEGF-α assay is carried out thanks to an ELISA test (PromoKine).

The results are shown in Table 2 below.

TABLE 2
Tests on HFDPC with evaluation of the synthesis activity of VEGF

| | Effective | Theoretical |
|---|---|---|
| | EC50 | EC50 |
| Ingredient (proportion) | (ppm) | (ppm) |
| Black Pepper Essential Oil | 38 | — |
| Cubeb pepper CO₂ extract | 25 | — |
| Pink berry CO₂ extract | 76 | — |
| Black Pepper/Cubeb mixture (1/1) | 16 | 30 |
| Black pepper/Pink berry mixture (1/1) | 30 | 51 |
| Cubeb/Pink berry mixture (1/1) | 24 | 38 |
| Cubeb/Pink berry/Black pepper mixture | 25 | 38 |
| (1/1/1) | | |

The effective EC50 is the extract concentration (in ppm) effective to obtain a VEGF synthesis activity which is 50% greater than the blank (i.e. culture without extract).

Theoretical EC50 is the EC50 value that would have been obtained in the absence of synergy between the extracts (average of the EC50 of the extracts in the mixture). It is obtained using a software which calculates it by extrapolation of the following equation for determining the IC50 of a mixture:

$$100 / {IC}_{50} mixture = \sum Ci / {IC}_{50} i .$$

$$in which$$

$$i = individual component of the mixture , varying from 1 to n$$

$$Ci = concentration of the component i$$

$$n = number of components in the mixture$$

$${IC}_{50} i = estimate of the {IC}_{50} of the component i .$$

The presented results attest to the effect, in particular the synergistic effect, of the extracts on the quantity of VEGF which is secreted by the treated cells and consequently on the hair growth.

1. A method for promoting the hair growth in a subject in need thereof, comprising the selection of a skin area on which the hair growth and/or regrowth is desired, and the application on said area of a composition, wherein the composition comprises at least one extract of Piper cubeba,
the extract of Piper cubeba is selected from the group consisting of CO₂ extract of Piper cubeba and essential oil of Piper cubeba.

2. The method of claim 1, wherein the extract of Piper cubeba is present in the composition in a sufficient quantity to increase VEGF synthesis activity.

3. The method of claim 1, wherein the extract of Piper cubeba represents at least 25 ppm by weight relative to the total weight of the composition.

4. The method of claim 1, wherein the extract of Piper cubeba represents from 25 ppm to 20% by weight relative to the total weight of the composition.
